(19) United States
(12) Patent Application Publication
(10) Pub. No.: US 2010/0113972 A1
(43) Pub. Date: May 6, 2010

(54) BIOPSY NEEDLE DEVICE AND METHOD FOR USING SAME

(76) Inventor: Victor Hugo Alvarado, San Jose (CR)

Correspondence Address: LARSON AND LARSON, 11199 69TH STREET NORTH, LARGO, FL, 33773, US

(21) Appl. No.: 12/262,978

(22) Filed: Oct. 31, 2008

(51) Int. Cl. A61B 10/02

(52) U.S. Cl. 600/567

(57) ABSTRACT

An improved biopsy needle includes a cannula having an open back end and a tip portion, the open back end is retained in a removeably fixed position by a biopsy gun. The cannula tip portion has a retractable cover for covering a cutting instrument when the cannula is inserted into the patient's body. The cutting instrument is integrally mounted along a lower edge of the cannula tip portion. A set of female threads is mounted along an inner circumference of the cannula at a distal end thereof, proximal to the cannula tip portion and the retractable cover. The retractable cover opens and exposes the cutting instrument when the cannula tip portion rotates and the retractable cover encloses the cutting instrument when the cannula tip portion is at rest. The cannula tip portion rotates in response to the biopsy gun firing a shot.

### FIELD OF THE INVENTION

The present invention relates to an improved biopsy needle and a method of using same. More particularly, it relates to an improved biopsy needle and method of using same, wherein the biopsy needle has a retractable tip portion for exposing a knife that cuts a tissue sample when entering a target area for sampling in a patient's body, the cut tissue sample entering a female threaded end of the needle for retracting from the body and for subsequent microscopic inspection thereof.

### BACKGROUND OF THE INVENTION

A biopsy procedure and the use of needles in biopsy procedures are both well known in the prior art. A biopsy procedure is a medical test of cells or tissue, typically from a suspicious lump, tumor or mass in a patient's body, which are removed from the body for examination by someone skilled in analyzing them, such as a pathologist. This examination is generally done under a microscope. However, certain chemical analysis procedures are also known and are employed in the prior art.

When a biopsy procedure takes only a tissue sample for examination, it is called an “incisional biopsy” or “core biopsy.” However, when an entire lump, tumor or mass is removed, it is called an “excisional biopsy” or “open biopsy.” Finally, when only a needle removes a tissue sample or fluid, the procedure is referred to as “a needle aspiration biopsy” or an “NAB.”

Needle aspiration biopsies are also referred to as “fine needle aspiration cytology,” or “FNAC, “fine needle aspiration biopsy,” or “FNAB,” or “fine needle aspiration” or “FNA.” Cytology refers to the study of cells. Needle aspiration biopsies are usually reserved for the investigation of lumps, tumors or masses that are superficial, or positioned directly under the skin. The procedure is considered a surgical procedure, albeit a minor one. However, it is a very useful procedure, especially when it rules out malignancy, since to do so can avoid the need for performing the more invasive and major surgical procedure of an excisional or open biopsy.

Needle aspiration biopsies are certainly less invasive and, therefore, less traumatic for a patient than an excisional or open biopsy. However, that is not to say that it is not uncomfortable or painful for a patient. In fact, typical complications and/or side effects are bruising of the skin, soreness and acute and localized pain at the point of sampling. Further, since typical needle aspiration biopsy procedures, and the known prior art devices used in such procedures, only take a small sample of cells, it is not uncommon to receive a false negative result or an indefinite diagnosis. The possibility of false negatives and indefinite diagnosis most often leads to the need for taking multiple samples from the patient. However, the taking of multiple samples, unfortunately, compounds the known complications and side effects which then causes additional and unwanted discomfort, soreness, pain and bruising for the patient. For all of these reasons, there is a need to improve upon existing needle biopsy devices to further limit the need for taking multiple samples, to improve definitive diagnosis, to reduce or eliminate false negatives, to limit or reduce patient complications and side effects, to take larger samples from smaller penetration depths, as well as to regulate the amount of pressure or force inflicted upon the patient when taking a biopsy sample.

Biopsy needles for use in needle aspiration biopsies are well known in the prior art. An early example of a prior art biopsy needle can be found in U.S. Pat. No. 2,705,949 to Silverman, wherein an outer tube is inserted into the targeted area (i.e., a lump, tumor or mass) and then a split needle is inserted therethrough for taking a tissue sample. Although this invention improved upon existing technology at that time by providing a means for limiting the advancement of both the outer tube and the inserted split needle, it still was extremely invasive and the user could not accurately and cleanly withdraw a biopsy sample of a certain length and size for subsequent study thereof, without causing a great deal of discomfort and pain to the patient. Further, the device only permitted one sample to be taken per insertion into the body and, therefore, had to be removed and sterilized after each insertion, regardless whether a sample was taken or not, before being re-inserted into the patient's body. Still further, there were an unneeded number of moveable parts to this device, which raised the probability of malfunction, total failure and false negative results and/or indefinite diagnosis.

Improvements to the device that is seen directly above, however embodying the same basic concept and technique for insertion, cutting and removal, can be seen in U.S. Pat. No. 4,784,156 to Garg. However, in this prior art reference, the biopsy needle includes an outer body (a hollow cannula) for insertion into the body, which is then used as a guide or securing a part for receiving an inner hollow needle instrument. The cannula has a distal tapered, circular sharp edge. The inner hollow needle instrument then comprises an inner solid stylet and an inner hollow cutting instrument. The cutting element includes a distal sharp edge cutting means. Although better and more precise samples can certainly be taken with this device, as compared to the earlier Silverman device, there are still too many working parts which make the device difficult to operate efficiently and accurately, and difficult to not cause pain and discomfort to the patient. Further, nothing in the Garg reference discloses, teaches or suggests that more than one sample can be taken at each insertion point. Further, this device is still fairly invasive because it requires a deep insertion for a tissue sample of workable study size. It should be remembered that small samples are more likely to lead to false negatives and/or an indefinite diagnosis, so therefore, larger samples are typically desired. This device certainly meets that need, but at the expense of the patient who must endure a painful and uncomfortable procedure inflicted upon his or her body. It should also be noted that although the Garg invention is certainly an improvement over the Silverman-type device, the use of an open sharp edge for cutting the sample remains an undesired feature of prior art biopsy needles, which adds to patient discomfort since the sharp edges continue to cut and slice tissue that will not be part of the sample during insertion and removal from the body. Among a host of other improvements that are needed, a shielded or closed cutting instrument that is deployable at time of the sample taking is highly desirable and needed in an improved biopsy needle.

Other improvements in the prior art, which include devices still used today, can be seen in U.S. Pat. No. 5,195,533 to Chin et al., which discloses a device with a side facing notch formed in a stylet. This device is similar to the well known Tru-Cut® device and is still widely used in the industry of biopsy procedures. Tru-cut® biopsy needles are disposable needles with an outer cannula and an inner, notched rod in which a tissue specimen is cut, trapped and withdrawn. In the referenced Chin et al. patent, however, the inventors were attempting to provide a device that could take multiple tissue samples without the need to completely remove the biopsy needle device. Although they may have been successful in this goal, the device still requires a deeper than necessary penetration into the sampled area of the patient's body (i.e., 10 mm of penetration for a 10 mm sample), and also employs the open sharp cutting instrument. Therefore, the subject referenced prior art device falls well short of overcoming most of the major deficiencies in the prior art, and is further known to cause unwarranted and unnecessary pain and discomfort to the patient by penetrating the body too deeply and by continuing to cut the tumor, mass or lump while entering and leaving the body, even though it is finished with taking its sample or samples.

A better example of an improved biopsy needle device can, therefore, be seen in U.S. Pat. No. 5,964,716 to Gregoire et al. In this referenced prior art device, the inventors provide a multi-ported piercing needle for taking multiple samples at a single sample site through the use of the multi-ported piercing needle. However, a vacuum is needed to extract each tissue sample and the patient must once again endure the painful and uncomfortable deep penetration required to take samples of significant length. Also, the intricate configuration and numerous parts of this particular prior art device make it prone to failure and requires an expert in its use to affect the necessary sample taking. Therefore, this device also falls short of overcoming most of the deficiencies of the prior art, and so further improvement is still needed.

Although not directly related to improving upon the devices mentioned above, there are a plurality of prior art devices that employ helical-like tip portions on auger-like devices on end portions of biopsy needle devices. However, in all of these devices, these tip portions are used in the localization of the device and not used for retaining a sample within a biopsy needle. Examples include U.S. Pat. No. 4,682,606 to DeCaprio and U.S. Pat. Nos. 5,018,530, 5,197,482 and 5,234,426 all to Rank et al. All of these helical tips are also movable within a cannula or needle and not integral with the inner surface of the cannula. This is an important distinction when compared to the present invention, which will be discussed in more full detail hereinafter. No prior art device uses a threaded female portion for receiving and retaining a tissue sample.

Prior art biopsy needles have fallen well short of providing any single device that can overcome all of the deficiencies seen within the industry. In particular, there is no single device that provides a minimally invasive needle device that can penetrate the patient's body at a depth less than the total length of the tissue sample. Further, no device can do so and take more than one sample. Still further, no such device exists having these above-mentioned revolutionary features which also incorporates a hidden, but deployable, cutting instrument on a penetrating tip portion that limits the unnecessary, and almost always painful, if not at least uncomfortable, cutting of patient tissue. And yet even further, no device in the prior art provides all of these needed features in a single device, while also permitting the user to choose a negative pressure within a chamber before taking a tissue sample to reduce or regulate the amount of force inflicted upon the patient when entering vital body organs or areas of great sensitivity or areas of differential mass.

All of these features are needed and, therefore, improvements upon existing prior art biopsy needle devices are clearly needed.

### SUMMARY OF THE INVENTION

I have invented an improved biopsy needle device that overcomes all of the aforementioned deficiencies seen in the prior art. In particular, I have invented an improved biopsy needle and device which can be used with a traditional biopsy needle gun apparatus, as commonly seen in the prior art, or which can be used alone.

My biopsy needle employs a thin, hollow cannula having a head portion for placement within a retaining portion. The cannula has a piercing tip located on an end portion distal from the head portion for penetrating the patient's body. A retractable cover, pivotable upon the cannula tip portion, opens by pressure when the tip portion rotates to take a sample, thereby alleviating any unwarranted cutting of body tissue when inserting the cannula within the body. The movement or opening of the self-retracting cover exposes a sharpened spoon-like cutting instrument for taking a first sample when the biopsy gun fires a shot. The sharpened spoon-like cutting instrument “scoops” a tissue sample, much like an ice-cream spoon scoops a portion of ice cream from its container. The retractable cover flips open in a preferred embodiment but can also slide within the tip portion when exposing the cutting instrument in an alternate embodiment. The cover closes after each shot, such that when the cannula is removed from the body, no cutting of tissue occurs.

Behind the cutting instrument is a threaded female portion integral with an inner surface of the cannula end portion. The threaded female portion receives a first sample therewithin when a first “shot” is taken by the biopsy needle device/gun. The cannula is mounted within the device so that it rotates with the rotational movement of a treaded retaining member within the device. A tensioned spring releases the threaded mounting member and permits the first shot to be taken. Since the end portion of the cannula is rotating while penetrating deeper within the sample area and taking the sample within the threaded female portion of the cannula, a sample having a length twice the depth of mass penetration is gathered. Therefore, by way of example, a 5 mm penetration shot from the biopsy gun will yield a 10 mm length tissue sample. This greatly reduces patient discomfort, all the while yielding and realizing a better usable tissue sample for examination. In the preferred embodiment, the cannula and needle tip rotates 360 degrees. A knuckle mounted along the outer circumference of the threaded retaining member engages a notch in the biopsy gun outer member (or elongated tubular member), which stops the forward motion of the threaded retaining member and, therefore, stops the forward movement of cannula. And since the cannula stops rotating, the cutting of tissue ceases as well.

After taking a first sample, if desired, the biopsy needle of the present invention can be relocated by slightly moving the device in any direction about the localized axis of penetration, but without the need of retracting the device, to take a second shot and to thereby extract a second sample. When the second shot is taken, the tensioned spring releases more of the held pressure from the spring and again rotates 360 degrees so that the cannula and tip portion again move forward and take the second sample within the inner female threaded cannula portion. The released tension of the spring can act as a forward stop member for the penetrating cannula or the knuckle can enter yet another notch in the biopsy gun outer member for stopping its forward motion. On the second shot, the depth of penetration again into the body is half the length of the tissue sample being taken from the body. The device is then removed with the cutting instrument closed during retraction.

Further, the improved biopsy needle device can adjust the amount of pressure that is used for one or both shots. This is important when entering a vital body organ, such as a lung, or an area of extreme sensitivity. This is accomplished by retracting a plunger inserted within a back end of the threaded retaining member. The distance of retraction is chosen by the operator of the device and/or by recommendation of the patient's doctor. By doing so, a chamber of negative pressure is formed within the threaded retaining member. So, when the first shot is taken, the distance of penetration of the cannula and the force of forward movement of the threaded retaining member is reduced by the backward pulling force of the negative pressure created within the chamber against the force of the released tension of the spring. The amount of negative pressure that can be introduced within the chamber is chosen after the cannula is introduced into the body and localized, but before the first shot is taken.

Therefore, an object of the present invention is to provide an improved biopsy needle device.

Another object of the present invention is to provide an improved biopsy needle device that takes needle aspiration biopsy samples.

Yet another object of the present invention is to provide an improved biopsy needle device that can take two biopsy samples without the need to retract and re-introduce the device into the patient's body.

Yet another object of the present invention is to provide an improved biopsy needle device that can yield a biopsy sample having a length twice as long as compared to the depth of penetration by the sampling-taking portion of the device, and to safely retain it within a cannula tip portion.

Still yet another object of the present invention is to provide an improved biopsy needle device that can regulate the amount of pressure used when a shot is taken.

Still yet another object of the present invention is to provide an improved biopsy needle device that can expose the sample-cutting instrument without inflicting unwarranted pain and/or discomfort to the patient upon introduction or retraction of the biopsy needle device in and out of the body.

Other objects of the present invention will become apparent when considering the below set forth detailed description of the drawings along with detailed description of the preferred embodiment.

### BRIEF DESCRIPTION OF THE DRAWINGS

This invention can be best understood by those having ordinary skill in the art by reference to the following detailed description, when considered in conjunction with the accompanying drawings in which:

FIG. 1 is a perspective view of a novel improved biopsy needle device of the present invention in a relaxed state or after a second shot has been taken;

FIG. 2 is a perspective view of the novel improved biopsy needle device in a tensioned state, which is ready to fire a first shot and take a tissue sample when inserted within a body;

FIG. 3 is an exploded view of the novel improved biopsy needle device of the present invention;

FIG. 4 is a cross-sectional view, along lines 4-4 of FIG. 2, illustrating the present novel device in a fully tensioned state and ready to fire a first shot;

FIG. 5 is a cross-sectional view, along lines 4-4 of FIG. 2, illustrating the present novel device in a partially tensioned state after firing a first shot, but ready to fire a second shot wherein a small chamber of negative pressure has been created by the forward movement of the threaded retaining member within the elongated tubular member;

FIG. 6 is a cross-sectional view, along lines 4-4 of FIG. 2, illustrating the present novel device in a fully relaxed state after firing a second shot;

FIG. 7 is a cross-sectional view, along lines 4-4 of FIG. 2, illustrating the present novel device in a fully tensioned state and ready to fire a first shot, but having a negatively pressurized chamber created behind the cannula within the thread retaining member for controlling the amount of pressure that will be inflicted upon the tissue mass of a body when the first shot is taken and, subsequently, for any second shot taken thereafter;

FIG. 8 shows a cannula tip portion of the present invention illustrating a closed cover position such that a cutting instrument contained thereunder is not exposed; and

FIG. 9 again shows the cannula tip portion of the present invention, but instead illustrating an open cover position such that the cutting instrument is exposed by a retractable cover portion flipping open when pressure is applied thereto, such as when the cannula rotates 360 degrees upon the device being fired.

### DETAILED DESCRIPTION OF THE PREFERRED EMBODIMENT

Throughout the following detailed description the same reference numerals refer to the same elements in all figures.

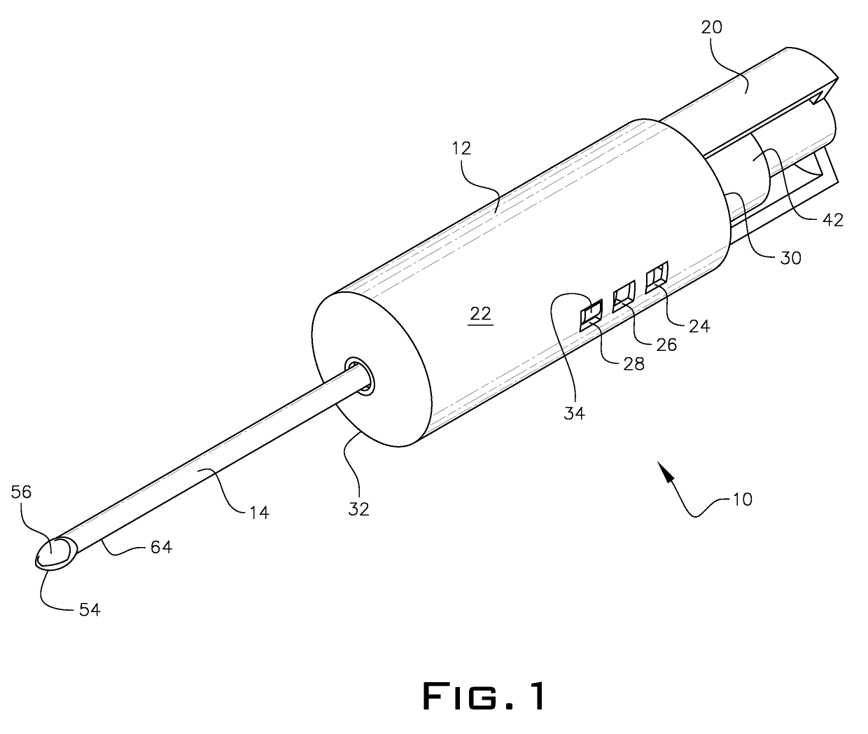

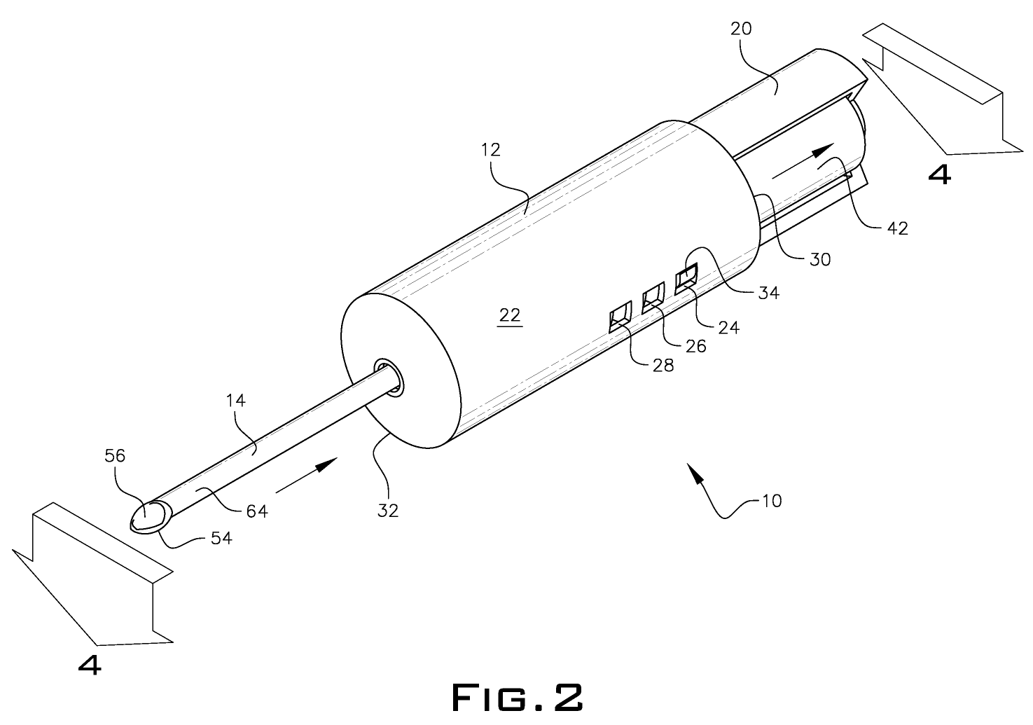

Referring to FIGS. 1-3, an improved biopsy needle device 10 of the present invention is shown. Device 10 has an elongated tubular member 12, which acts as a retaining member for a cannula 14, a threaded retaining member 16, a tensionable spring 18 and a plunger element 20 (all seen in the exploded view of FIG. 3). Cannula 14 is actually further retained by threaded retaining member 14, as shown in FIG. 3. With reference to FIG. 1, device 10 is in a relaxed state such that spring 18 would be fully extended or at least extended to its full permissible limit. FIG. 2 on the other hand, illustrates device 10 in its fully tensioned state, which is also considered its “ready to fire” a first shot state.

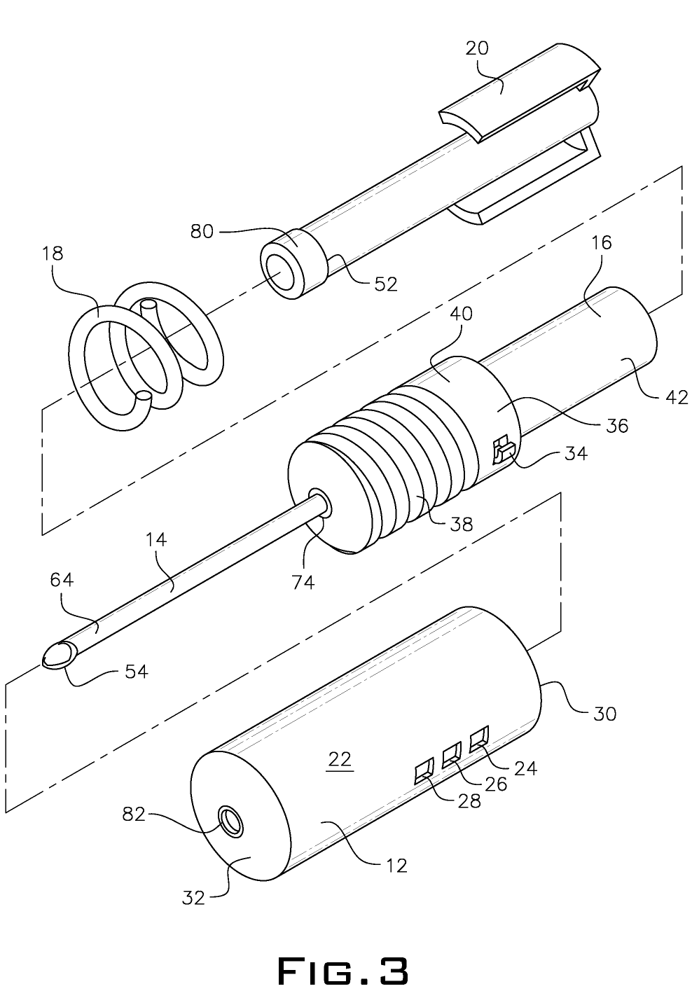

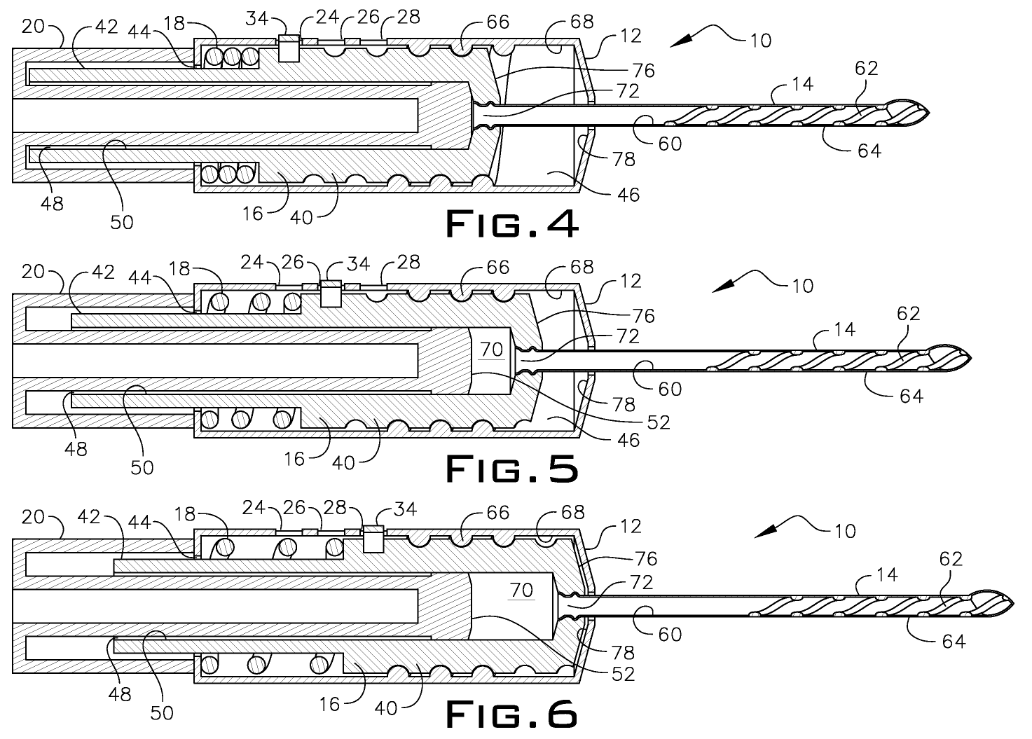

With continuing reference to FIGS. 1-3, an outer circumference 22 of elongated tubular member 12 has a series of notches formed therein. In the preferred embodiment, three notches are employed, although nothing herein limits the use of more or less than three notches. As to the preferred embodiment, a first notch 24 is proximal to a back end 30 of the device 10, whereas a second notch 26 is positioned intermediate first notch 24 and a third notch 28, which is then distal to device back end 30 but proximal to a front end 32 of device 10. Notches 24, 26 and 28 are used as stops for device 10 by receiving a knuckle 34 formed along an outer circumference 36 of an upper barrel member 40 of threaded retaining member 16 (as shown in FIG. 3), when threaded retaining member 16 rotates within a cavity 46 (see FIGS. 4, 5 and 7) of elongated tubular member 12. As shown in the preferred embodiment, notches 24, 26 and 28 are axially aligned along outer circumference 36 of upper barrel member 40. However, nothing herein limits different configurations and positionings for the notches, such as, by way of example, an offset or angled configuration.

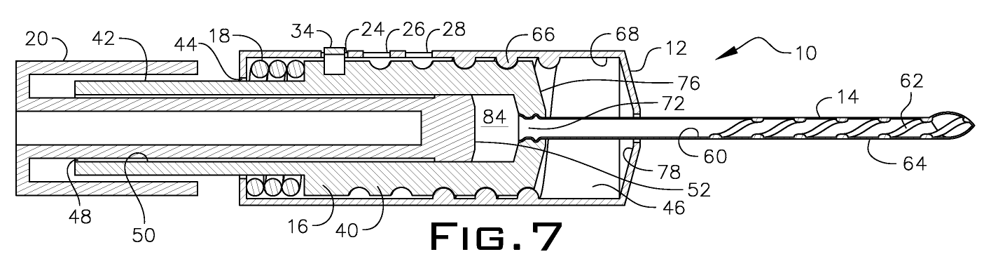

As shown in FIG. 3, knuckle 34 is positioned slightly behind a set of threads 38 of upper barrel member 40 of threaded retaining member 16. Threaded retaining member 16 also includes an integral lower barrel member 42, which contains an open back end 44 (see FIGS. 4-7) for receiving plunger element 20 therewithin. Upper barrel member 40 has a radius that is greater than that of lower barrel member 42 (which can also be seen in FIGS. 4-7). Further, threaded retaining member 16 has its own cavity 48 that has an equal circumference along an inner wall 50 of the entire threaded retaining member 16 through both lower and upper barrel members 42 and 40, respectively (see FIGS. 4-7). Cavity 48 of threaded retaining member 16 is used to create negatively pressurized chambers therewithin and in front of an end portion 52 of plunger element 20, either as a result of firing device 10 or by operator choice before firing device 10 (to be discussed in further detail hereinbelow and as illustrated in FIG. 7).

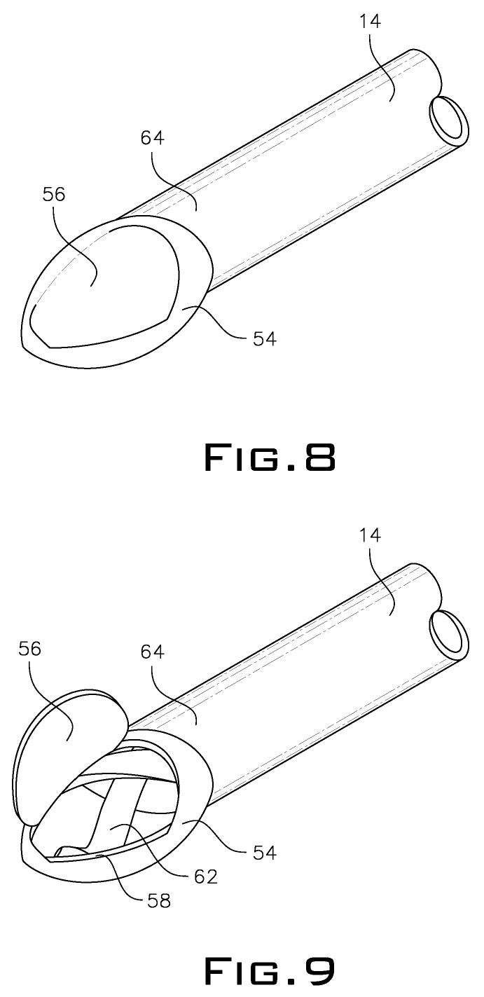

Referring now to FIGS. 8 and 9, a tip portion 54 of cannula 14 is shown in two different states, opened (FIG. 9) and closed (FIG. 8). A retractable cover 56 exposes a sharpened tissue cutting instrument 58 positioned along a bottom edge of tip portion 54 when retractable cover 56 is open. Retractable cover 56 opens in response to pressure applied thereto, such as when cannula tip portion 54 rotates within the body (see FIGS. 8 and 9). Likewise, retractable cover 56 closes in response to a lack of pressure, such as when cannula tip portion 54 stops rotating. When retractable cover 56 opens, it flips outward away from tip portion 54 as shown in FIG. 9. However, in an alternate embodiment (not shown), cover 56 could slide inward within cannula tip portion 54. Further, device 10 can be made to rotate clockwise or counter-clockwise and is dependent upon whether a “flip-out” or a “slide-in” cover is employed.

Referring now to FIGS. 4-7, it is shown that cannula 14 has an inner circumference 60 on which a set of female threads 62 are formed along a distal end portion 64, thereof. Female threads 62, which can also be seen in FIG. 9, are for receiving a tissue sample after cutting instrument 58 has sliced a mass inside of a body after retractable cover 56 has opened and after tip potion 54 of cannula 14 has made a turn, which in the preferred embodiment is 360 degrees (as represented in FIG. 9). However, nothing herein limits the turning of cannula tip portion 54 from less than 360 degrees. Also, to the preferred embodiment, female threads 62 cover about 10 mm of length of cannula distal end portion 64, although other lengths of varying size can be used.

Referring to FIG. 3, and with reference to FIGS. 4-7, as well, it is shown that spring 18 inserts over lower barrel member 42 of threaded retaining member 16. In a fully tensioned state, and being ready to fire a first shot, spring 18 is fully compacted, as shown in FIG. 4. Presuming that device 10 is inserted into a body, a first shot is taken whereby spring 18 pushes threaded retaining member 16 forward by rotating along a set of reciprocal receiving threads 66 of an inner circumference 68 of elongated tubular member 12. This causes retractable cover 56 to open and expose cutting instrument 58 as cannula tip portion 54 rotates. As previously explained, and as shown between FIGS. 4 and 5, knuckle 34 retracts from first notch 24 and engages second notch 26. Because of the first shot, and as shown in FIG. 5, a small chamber of negative pressure 70 is formed between plunger end portion 52 and an open back end 72 of cannula 14, which is held in place by a receiving aperture 74 (see FIG. 3) of threaded retaining member 16. Also, because of the first shot and because cannula tip portion 54 rotates 360 degrees (as shown in FIG. 9), a tissue sample is drawn within cannula inner circumference 60 along female threads 62. Because in the preferred embodiment cannula tip portion 54 rotates 360 degrees, and because of the spiral placement of female threads 62 within cannula inner circumference 60, cannula 14 can advance a certain distance (or depth) x, but cut (or remove) a tissue sample having a length of 2 x. By way of example, if cannula 14 advances 5 mm during the first shot, then cutting instrument 58 and cannula tip portion 54 cut and take a 10 mm sample therewithin.

After the first shot, spring 18 is still tensioned and ready to move forward again. Therefore, a second shot can be taken, whereby again knuckle 34 disengages from second notch 26 which permits threaded retaining member 16 to rotate another 360 degrees around reciprocal receiving threads 66 of elongated tubular member inner circumference 68 until knuckle 34 engages third notch 28, as shown between FIGS. 5 and 6. This adds to the chamber of negative pressure 70, formed between plunger end portion 52 and open back end 72 of cannula 14. In addition to the knuckle 34 and third notch 28 engaged activity, the released tension of spring 18 and the positioning of an outer end wall 76 of threaded retaining member 16, which has reached an inner end wall 78 of elongated tubular member 12 (as seen in FIG. 6), acts as additional stops for device 10 after the second shot has been taken. The result of the second shot provides for another sample taken within cannula inner circumference 60 along female threads 62. And again, the length of the tissue sample is 2 x if the distance of advancement (or depth of penetration) of cannula 14 is x. It should be noted, though, that the rotation of threaded retaining member 16 of 360 degrees is employed in the preferred embodiment. Nothing herein limits the use of rotations of lesser amounts, such as 90 degrees, wherein notches 24, 26 and 28 are then set at an angle to one another or off-set, as described hereinabove.

Referring to FIG. 3, it is shown that plunger element 20 has a small outer sealing member 80 mounted on plunger element end portion 52. This acts as an air seal within cavity 48 of threaded retaining member 16 and creates the negative pressurized chambers 70 after the first and second shots are taken.

In some situations, the operator of device 10 may desire to reduce the amount of pressure exerted upon the patient's body when firing each the first and second shot. Therefore, as shown in FIG. 7, it is possible to withdraw plunger element 20 slightly backwardly, whereby a pre-firing negatively pressurized chamber 84 is formed. The existence of pre-firing negatively pressurized chamber 84 results in less pressure being exerted for each shot since the negatively pressurized chamber pushes back against the air entering cannula 14 for each shot, and thereby causes cannula 14 to exert less force. This is most often used when taking a tissue sample from a very sensitive area or a soft mass, which requires less force for taking said sample. However, in this use and embodiment, the first and second shots are taken in the same manner as described hereinabove, and therefore, the tissue samples are taken in the same way as described before in the description above.

Other equivalent elements can be substituted for the elements disclosed herein to achieve the same results in the same way and in the same manner.

Having thus described the invention, what is claimed for Letters Patent follows:

1. An improved biopsy needle for insertion within a body of a patient for taking at least one cellular sample, the improved biopsy needle inserted into a biopsy needle gun for taking said at least one cellular sample when the biopsy gun fires at least one shot, the improved biopsy needle comprising:
a) a cannula having an open back end and a tip portion, the open back end retained in a removeably fixed positioned by the biopsy gun;
b) the cannula tip portion having a retractable cover for covering a cutting instrument when the cannula is inserted into the patient's body, the cutting instrument integrally mounted along a lower edge of the cannula tip portion;
c) a set of female threads mounted along an inner circumference of the cannula, at a distal end thereof, proximal to the cannula tip portion and the retractable cover, the retractable cover opening and exposing the cutting instrument when the cannula tip portion rotates, the retractable cover covering the cutting instrument when the cannula tip portion is not rotating;
d) the cutting instrument cutting the at least one cellular sample when the cannula tip portion is rotating and the cutting instrument is exposed;
e) the set of female threads guiding the at least one cellular sample into the cannula and retaining it therewithin; and
f) the cannula tip portion rotating in response to the biopsy gun firing a shot.

2. The improved biopsy needle of claim 1, wherein the length of the at least one cellular sample is approximately twice as long as a depth of penetration of the cannula within the patient's body when the biopsy gun fires the at least one shot.

3. The improved biopsy needle of claim 1, wherein the cannula retains first and second different cellular samples in response to the firing of the biopsy gun first and second times.

4. A biopsy needle device comprising:
a) a cannula having an open back end and a tip portion, the open back end removeably held within an aperture of a threaded retaining member, the threaded retaining member having a set of threads mounted along an outer circumference of an upper barrel, a lower barrel integrally attached behind the upper barrel and an inner cavity formed through both the upper and lower barrels;
b) an elongated tubular member having an inner cavity formed therein for receiving the threaded retaining member, the elongated tubular member having a reciprocal set of threads formed along an inner circumference of its inner cavity for engaging the threaded retaining member when inserted therein, the elongated tubular member having an aperture formed in a front end, which is in axial alignment with the threaded retaining member aperture for permitting the cannula to protrude out through the elongated tubular member.
c) a spring inserted over the threaded retaining member lower barrel and held within the elongated tubular member inner cavity at a back end thereof, the spring pushing the threaded retaining member forward when tension thereof is permitted to be released;
d) a plunger element inserted within the threaded retaining member inner cavity, the plunger element having an air sealing plug mounted about its distal end;
e) a cutting instrument mounted along an edge portion of the cannula tip portion;
f) a set of female threads mounted on an inner circumference of a distal end of the cannula, the cutting instrument and the set of female threads for cutting, taking and retaining a cellular tissue sample from a patient's body; and
g) means for firing the biopsy needle device at least one time.

5. The biopsy needle device of claim 4, further comprising a retractable cover mounted at the cannula tip portion for covering the cutting instrument when not taking a cellular tissue sample from the patient's body.

6. The biopsy needle device of claim 5, wherein the retractable cover opens and exposes the cutting instrument in response to the cannula tip portion rotating within the patient's body.

7. The biopsy needle device of claim 6, wherein the retractable cover flips open and outwardly to expose the cutting instrument and then flips back downwardly thereby covering the cutting instrument when the cannula tip portion is not rotating within the patient's body.

8. The biopsy needle device of claim 6, wherein the cannula tip portion rotates in response to firing the biopsy needle device.

9. The biopsy needle device of claim 8, wherein the cannula tip portion generally rotates between 90 and 360 degrees each time that the biopsy needle device is fired.

10. The biopsy needle device of claim 4, wherein the means for firing the biopsy needle device at least one time comprises a knuckle mounted along an outer circumference of the threaded retaining member upper barrel removeably engaged within a notch of an at least one notch formed in the elongated tubular member, the threaded retaining member rotating within the elongated tubular member when the knuckle disengages from the notch and the spring releases its tension.

11. The biopsy needle device of claim 10, wherein the at least one notch includes a first, second and third notch.

12. The biopsy needle device of claim 11, wherein the first, second and third notches are formed and axially aligned along the elongated tubular member.

13. The biopsy needle device of claim 11, wherein the first, second and third notches are formed in an offset position respective to one another along the elongated tubular member.

14. The biopsy needle device of claim 4, wherein the biopsy needle device can be fired two times such that the cannula tip portion rotates two times thereby permitting the cutting instrument to take two separate tissue cellular samples, each of the two separate tissue cellular samples retained within the cannula distal end within the female threads mounted along the inner circumference thereof.

15. The biopsy needle device of claim 4, wherein a chamber of negatively pressurized air is formed in front of the plunger element air sealing plug and the cannula open back end when the biopsy needle is fired.

16. The biopsy needle device of claim 4, wherein a pre-firing negatively pressurized air chamber is formed in front of the plunger element air sealing plug and the cannula open back end by partially withdrawing the plunger element from within the threaded retaining member cavity.

17. The biopsy needle device of claim 4, wherein the cellular tissue sample that is cut and taken by the cutting instrument, and which is retained by the set of female threads within the distal end of the cannula, has a length that is twice as long as a depth of penetration of the cannula tip portion after advancing in the patient's body in response to a shot being fired by the biopsy needle device.

18. A method of taking a cellular tissue sample from a patient's body using a fine needle aspiration biopsy needle, the steps of the method comprising:
a) providing a biopsy needle having a cannula, the cannula having an open back end and a tip portion, the tip portion having a retractable cover that opens in response to tip portion rotation and closes in response to the tip portion being at rest, the tip portion also having a cutting instrument mounted along a lower edge, the cutting instrument exposed when the retractable cover is open and covered when the retractable cover is closed, the cannula further having a female threaded portion integral with a inner circumference of the cannula at a distal end but proximal to the cannula tip portion;
b) providing a biopsy needle gun having a retaining cavity for a biopsy needle and a means for firing at least one shot;
c) mounting the biopsy needle into the biopsy needle gun;
d) inserting the biopsy needle mounted within the biopsy needle gun into a target area of the patient's body;
e) engaging the biopsy needle gun means for firing at least one shot;
f) firing the biopsy needle gun means for firing at least one shot such that the cannula tip portion rotates and the retractable cover opens;
g) cutting a cellular tissue sample with the cutting instrument;
h) retaining the cellular tissue sample within the cannula around the female threaded portion;
i) stopping the rotation of the cannula tip portion such that retractable cover closes; and
j) extracting the biopsy needle mounted within the biopsy needle gun from the target area of the patient's body.

19. The method of taking a cellular tissue sample from a patient's body using a fine needle aspiration biopsy needle according to claim 18, further comprising the steps of:
a) relocating the tip portion of the biopsy needle cannula in the target area after firing the biopsy needle gun means for firing at least one shot but before extracting the biopsy needle mounted within the biopsy needle gun from the target area of the patient's body;
b) re-engaging the biopsy needle gun means for firing at least one shot;
c) firing the re-engaged biopsy needle gun means for firing at least one shot such that the cannula tip portion rotates again and the retractable cover re-opens;
d) cutting a second cellular tissue sample with the cutting instrument;
e) retaining the second cut cellular tissue sample within the cannula around the female threaded portion; and
f) stopping the rotation of the cannula tip portion such that retractable cover closes.

20. The method of taking a cellular tissue sample from a patient's body using a fine needle aspiration biopsy needle according to claim 18, further comprising the steps of:
a) providing a plunger element inserted within an inner cavity of a retaining member of the biopsy needle gun, the plunger element having an air sealing plug mounted about its distal end; and
b) withdrawing the plunger element partially from the inner cavity of the biopsy needle gun retaining member thereby forming a pre-firing negatively pressurized air chamber between the plunger element distal end and the cannula open back end, the partial withdrawing of the plunger element occurring before the step of firing the biopsy needle gun means for firing at least one shot.
